(19) United States
(12) Patent Application Publication
(10) Pub. No.: US 2008/0171707 A1
(43) Pub. Date: Jul. 17, 2008

(54) Use Of An Onion Extract For Making A Composition To Control Weight Gain

(75) Inventors: Marie-Josephe Amiot-Carlin, Montfavet (FR); Christine Juhel, Saint-Cyr Sur Mer (FR); Frederic Tosini, Bandol (FR); Louis Cara, Marseille (FR); Veronique Berger, Lyon (FR)

Correspondence Address: YOUNG & THOMPSON, 209 Madison Street, Suite 500, ALEXANDRIA, VA, 22314, US

(73) Assignee: Scalime Nutrition, Avignon (FR)

(21) Appl. No.: 11/908,684

(22) PCT Filed: Mar. 14, 2006
(86) PCT No.: PCT/FR2006/050224

§ 371 Date: Dec. 19, 2007

(30) Foreign Application Priority Data
Mar. 14, 2005 (FR) 0550650

(51) Int. Cl. A61K 31/7016; A61K 31/352; A61K 31/7004

(52) U.S. Cl. 514/23; 514/456

(57) ABSTRACT

The invention concerns the use of an onion extract comprising 20 wt. % to 40 wt. % of polyphenols of the flavonol family, said onion extract having the following composition characteristics, expressed in weight relative to the total extract dry weight: 60 wt. % to 90 wt. % of the total flavonols of said extract consist of glycosylated polyphenols; 85 wt. % to 98 wt. % of total flavonols of said extract consist of free-form (aglycon) or glycosylated quercetin; 5 wt. % to 20 wt. % of the total flavonols of said extract consist of quercetin-3,4′-diglucoside; 30 wt. % to 70 wt. % of the total flavonols of said extract consist of quercetin-3-monoglucoside and quercetin-4′-monoglucoside; and 20 wt. % to 30 wt. % of the total flavonols of said extract consist of quercetin, for making a composition to control weight gain.

The present invention relates to the field of nutritional compositions and pharmaceutical compositions intended to regulate or to control the weight gain.

Excess body weight represents a problem having an increasing incidence on the human health in the whole developed or rapidly industrializing countries in the world.

In the United States, approximately 61% of the adult individuals have an excess weight, which is expressed as a body mass index (BMI) higher than 25 kilos per square meter. The results of the NHANES III study dating from 1988 to 1994 show that 20% of men and 25% of women suffer from obesity (BMI>30)

In France as well the overweight or obesity incidence frequency is increasing for both sexes. In July 2004 the AFSSA published data revealing that 19% of children are obese. The ObEpi/SOFRES study in 2003 showed that 11.3% of adults are obese (BMI>30) and 41.6% of adults are overweight (25<BMI<29.9). Excess weight and obesity mainly result from a food imbalance and from insufficient physical training and are both associated with a plurality of diseases such as some cancers, cardiovascular diseases and diabetes.

According to the World Health Organization (WHO), the excess weight and obesity are the most frequently encountered chronic diseases in children and young persons living in the leading industrialized countries, including Europe and the United States. Thus, according to the World Health Organization, there are more than 300.000 obese adults in the world.

The environmental and behavioural changes accompanying economical development, modernization and urbanization are associated with the overall obesity increase in adults and children.

The body weight increase results from an imbalance between energy supply and energy expenditure in the individual and does express as an excessive adipose tissue mass expansion. The excess weight, including obesity, increases the risk of hypertension, of type 2 diabetes, of arthritis, of hyperlipidemia high levels, of cancer of high-risk pregnancy or of asthma.

Dietetic approaches to regulate the excess body weight have known a limited success. Low-calorie diets may cause a temporary weight loss, but they failed to demonstrate they could be a long term solution for the persons wishing to loss weight, and then to maintain such lower weight.

The appetite suppressants, that do reduce the food intake, that do increase energy expenditure and/or modify the metabolism have a potential efficacy to reduce the weight gain. Unfortunately, these drugs are often associated with various adverse drug reactions, amongst which some could be mortal in a few exceptional situations.

In the art, there is therefore a need for new compositions enabling to control the weight gain, that would be efficient, that would present less adverse reactions and that would be cost effective.

Such compositions enabling to control the weight gain in humans or animals are provided according to the present invention. In practice, the compositions of the invention are used by individuals that preferably will simultaneously undergo a food intake control.

Surprisingly, it has been shown according to the present invention that an onion purified extract having a high content of polyphenolic compounds presents a great efficiency in limiting the weight gain of a mammal consuming a lipid-rich, atherogenic diet.

It is a object of the present invention to provide the use of an onion extract comprising from 20% to 40% by weight of polyphenols belonging to the flavonol family, said onion extract having following composition characteristics, expressed by weight based on the dry extract total weight.
from 60% to 90% by weight of the total flavonols in said extract consist in glycosylated flavonols,
from 85% to 98% by weight of the total flavonols in said extract consist in quercetin in a free form (aglycone) or in a glycosylated form,
from 5% to 20% by weight of the total flavonols in said extract consist in quercetin-3,4′-diglucoside,
from 30% to 70% by weight of the total flavonols in said extract consist in a mixture of quercetin-3-monoglucoside and quercetin-4′-monoglucoside, and
from 20 to 30% by weight of the total flavonols in said extract consist in quercetin,
   for preparing a weight gain control composition.

As used herein, “quercetin” and “quercetin glycosides” represent compounds having following formula (I):

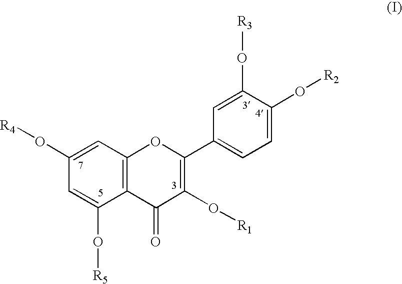

wherein:

R₁ to R₅ groups represent independently from each other an oside residue or a polyoside chain having from 1 to 3 oside units, or a hydrogen atom.

The quercetin compound in its free form (aglycone) is a compound of formula (I), wherein the R₁ to R₅ groups each represent a hydrogen atom.

The quercetin-3,4′-diglucoside compound is the compound of formula (I), wherein the R₁ and R₂ groups each represent a glucose oside residue and the R₃, R₄ and R₅ groups each represent a hydrogen atom.

The quercetin-3-monoglucoside compound is the compound of formula (I), wherein the R₁ group represents a glucose oside residue and the R₂, R₃ and R₄ groups each represent a hydrogen atom.

The quercetin-4′-monoglucoside compound is the compound of formula (I), wherein the R₂ group represents a glucose oside residue and the R₁, R₃, R₄ and R₅ groups each represent a hydrogen atom.

The quercetin glycoside compounds that may be contained in the onion extract such as defined hereabove include in particular quercitrin, which is a compound of formula (I), wherein the R₁ group represents a 6-desoxy-α-L-mannopyranosyl oside residue, and the R₂, R₃, R₄ and R₅ groups represent a hydrogen atom.

The compounds that may be contained in the onion extract such as defined hereabove also include rutin, which is a compound of formula (I), wherein the R₁ group represents a rutinose group, and the R₂, R₃, R₄, and R₅ groups represent a hydrogen atom. As is well known, the rutinose group is a disaccharide formed with two oside residues, the rhamnose (also called 6-deoxy-L-mannose) and the glucose, respectively.

Suitable examples of quercetin glycosides that may be contained in the onion extract such as defined hereabove include 3-O-β-D-glucosyl-(1→2)-β-D-glucosyl-(1→2)-beta-D-glucosyde quercetin, wherein the R₁ group represents the β-D-glucosyl-(1→2)-beta-D-glucoside group and the R₂, R₃, R₄ and R₅ groups represent a hydrogen atom.

Quercetin glycosides that are compounds of formula (I) wherein one of the R₁, R₂, R₃, R₄ and R₅ groups, but more specifically the R₁ group represents one of the oside groups selected from α-L-(5″-O-acetyl)-arabinofuranose, α-L-arabinofuranose, α-L-(3″-O-acetyl)-arabinofuranose, or α-L-rhamnopyranose are also suitable.

Another flavanol is the isorhamnetin compound that is a compound of formula (I), wherein the R₃ group represents a methyl (CH₃) group and the R₁, R₂, R₄ and R₅ groups each represent a hydrogen atom.

Another flavanol is the compound 3,4′-isorhamnetin quercetin, that is the compound of formula (I), wherein the R₃ group represents a methyl (CH₃) group, the R₁ and R₂ groups each represent a glucose oside residue and the R₄ and R₅ groups each represent a hydrogen atom.

Another flavanol is the compound isorhamnetin-4′-monoglucoside compound, that is the compound of formula (I), wherein the R₃ group represents a methyl (CH₃) group, the R₂ group represents a glucose oside residue and the R₁, R₄ and R₅ groups each represent a hydrogen atom.

Kaempferol is another flavonol that may be contained in the onion extract such as defined hereabove, typically in a concentration of less than 1% by weight, as compared to the dry extract total weight.

As used herein, “kaempferol” represents the compound having following formula (II):

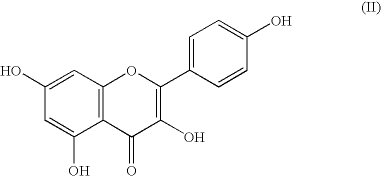

Generally speaking, for the onion extract composition characteristics, the amounts are expressed in 3′monoglucoside quercetin or isoquercitrin equivalents, for the glycosylated flavonols, or in quercetin equivalents for quercetin and for other aglycone flavonols.

As used herein, an “equivalent” is defined as an external standard for quantifying, assuming that the response factor of the dose compound is comparable to the response factor of that standard.

Thus, it has been demonstrated according to the invention that an onion purified extract, rich in polyphenols belonging to the flavonol family, of which from 85% to 98% by dry weight of the flavonols consist in a quercetin or quercetin glycosides, has a weight gain limitation action for mammals consuming a lipid-rich diet said to be “atherogenic”.

The present invention surprisingly also demonstrated that the purified, polyphenol-rich onion extract, in particular rich in quercetin and in quercetin glycosides, such as defined hereabove, does possess a weight gain limitation activity comparable to that of the ([2S-[2-α(R), 3β]]-N-formyl-L-leucine 1-[(3-hexyl-4-oxo-2-oxetanyl)methyl]dodecyl ester) tetrahydrolipstatin. Tetrahydrolipstatin is a very potent active ingredient used for treating obesity in combination with a low-calorie diet, and for treating non insulin-dependent diabetes.

Tetrahydrolypstatin is an active ingredient that does specifically inhibit the pancreatic lipase, which results for the human species in a reduction of about one third of the lipids contained in food on average, thus leading to a weight loss.

However, tetrahydrolipstatin is associated with some side effects, such as steatorrhoea, anal exudation, abdominal pains, nausea and vomiting, or fat-soluble vitamin absorption reduction.

In addition, it has also been demonstrated according to the invention that the polyphenol-rich, onion purified extract, in particular quercetin- and quercetin glycoside-rich onion purified extract, such as defined hereabove, has a weight gain limitation activity far greater than that of another polyphenol-rich plant extract, more specifically a polyphenol-rich grape-cake extract. It is known that grape total polyphenols make it possible to reduce cholesterolaemia by 9.7% after twelve weeks of atherogenic diet (Auger C, Gerain P, Laurent-Bichon F, Portet K, Bornet A, Caporiccio B, Croc G, Teissedre P L, Rouanet J M; 2004, J. Agric. Food Chem. K, vol. 52(16): 5297-5302).

In a first preferred aspect of the invention, from 70% to 80% by weight of the dry extract total polyphenols in the purified onion extract such as defined hereabove consist in glycosylated flavonols.

In a second preferred aspect of the invention, from 90% to 95% by weight of the flavonols in said dry extract consist in quercetin or quercetin glycosides.

In a third preferred aspect of the invention, from 7% to 13% by weight of the total flavonols in said dry extract consist in quercetin-3,4′-diglucoside.

In a fourth preferred aspect of the invention, from 40% to 60% by weight of the total flavonols in said dry extract consist in quercetin-3-monoglucoside and quercetin-4′-monoglycoside.

In a fifth preferred aspect of the invention, said purified onion extract comprises from 25% to 35% by weight of polyphenols belonging to the flavonol family in the dry extract.

The polyphenol content analysis, in particular of flavonols, in a purified onion extract such as defined hereabove may be conducted by the man skilled in the art by means of any suitable separation method known per se. For example, the man skilled in the art may conduct the polyphenol content analysis in a purified onion dry extract such as defined hereabove by means of various analytic methods including chromatography methods described by Baranowski and al. (Baranowski R, Kabut J., Baranowska I, 2004, Analytical Letters, vol. 37 (1):157-165), of Degenhardt and al. (Degenhardt A, Engelhardt U H, Lackenbrink C, Winterhalter P, 2000, J. Agric. Food Chem., vol. 48 (8): 3425-3430), of Crozier and al. (Crozier A, Burns J., Aziz A. A, Stewart A J Rabiasz H S, Jenkins G I, Edwards C A and Lean M E J, Biol. Res., Vol. 33 (2): 79-88) and of Guillen and al. (Guillen D A, Barroso C G, Perez-Bustramante J A, 1996, Journal of Chromatography A; vol. 724 (1): 117-124). The man skilled in the art may also use polyphenol separation methods based on capillary electrophoresis, such as described in particular by Pazourek and al. (Pazourek J., Gonzalez G, Revilla A L, Havel J, 2000, Journal of Chromatography A., vol. 874 (1): 111-119).

Polyphenol content analysis of an onion extract such as defined hereabove may also be conducted using the following method:

Phenolic compounds are separated by HPLC on an Alltima column of 5 μm (150×4.6 mm, Alltech) protected with a C18 Alltima precolumn with a gradient based on two solvents A (H₂O+HCOOH 0.05%), B (CH₃CN), at 0 min, 10% B; at 40 min, 40% B; at 50 min, 100% B. The flow rate is 1 mL·min⁻¹ and separation occurs at 35° C. The injected volume is 20 μl. Detection is made by spectrophotometry at 365 nm. Compounds are quantified using an external calibration. Amounts are expressed in isoquercitrin equivalents or in quercetin-3′-monoglucoside (QMG) equivalents for the quercetin glycosides or in quercetin equivalents for the other aglycone flavonols.

Most preferably, the onion purified dry extract such as defined hereabove presents, as a base component of a composition according to the invention, in a powdered form.

Most preferably, the hereabove purified dry onion extract rich in polyphenols belonging to the flavonol family is obtained by carrying a method for extracting, fractioning and purifying the polyphenolic compounds derived from onions, comprising the following steps:

a) extracting the polyphenolic compounds to obtain a raw onion extract;

b) adsorbing on an adsorbing resin the polyphenolic compounds contained in the raw extract;

c) eluting the polyphenolic compounds retained onto the resin to obtain a purified extract;

d) concentrating, then optionally drying the purified extract to obtain a product rich in polyphenolic compounds.

Most preferably, the adsorption step b) is performed on a styrene-divinylbenzene type resin, and more preferably a resin of this type having the following physical characteristics:

1) pores having a mean size of from 50 to 110 Angstroms, preferably of from 60 to 100 Angstroms,

2) a specific surface equal to or higher than 800 m²/g, preferably equal to or higher than 880 m²/g,

3) a pore volume higher than 1 ml/g, and preferably equal to or higher than 1.4 ml/g.

Most preferably, the extraction step a) comprises a step of rapidly heating the onion vegetable matter, for example the onion grade-out products, starting from an ambient temperature of 25° C. up to a temperature of 105° C. so as to obtain a hot exudation juice and a cooled exudated cooked onion pulp, the exudation juice forming at least partly the raw extract, then a step consisting in putting under vacuum the onion vegetable matter, thus leading to the vaporization of part of it, this step being conducted after the heating step, under a pressure ranging from 10³ to 2×10⁴ Pa abs.

Most preferably, the adsorption step b) is performed with a styrene-divinylbenzene type resin selected from resins marketed by Resindion under the trade names SP70 and SP700.

A particularly preferred method for preparing the onion purified dry extract used according to the invention is the method described in the PCT application N^{o} WO 02/064536, to which complete content the man skilled in the art will advantageously refer.

According to a particularly preferred aspect of the invention, for preparing a weight gain control composition, an onion purified dry extract such as defined hereabove will exclusively be used as physiologically active material on the weight gain.

In particular, in this preferred aspect of the invention, there will be for preparing a weight gain control composition no additional vegetable matter other than the purified onion dry extract as defined in the present description.

In particular, in one of its preferred aspects, the final composition for controlling the weight gain to be prepared does not contain any additional compounds enabling the re-uptake of neurotransmitters such as norepinephrine, serotonin or dopamine.

Moreover, in the most preferred aspect of the invention the final composition for controlling the weight gain to be prepared does not contain any additional compound having an effect on the fat, cholesterol or triglyceride adsorption and synthesis, as for example the hydroxycitric acid.

In particular, in the most preferred aspect of the invention, the final composition for controlling the weight gain to be prepared does not contain any additional compound of the polyphenol type, in particular of the flavonol type, and even more particularly of the quercetin or quercetin glycoside type, other than those already present in the onion purified dry extract such as defined in the present description.

According to a first embodiment, the onion purified dry extract such as defined hereabove is used for preparing a nutritional or a food composition intended to control the weight gain.

According to a second embodiment, the onion purified dry extract such as defined hereabove is used for preparing a pharmaceutical composition intended to control the weight gain.

### Nutritional Compositions of the Invention

Preferably, a nutritional composition that is prepared according to the invention comprises an amount of onion purified dry extract adapted to a daily oral administration of said onion extract ranging from 0.1 g to 10 g.

For a composition intended to the human consumption, a nutritional composition according to the invention comprises an amount of onion purified dry extract such as defined hereabove adapted to a daily intake of said extract, provided by said composition, ranging from 0.7 g to 5 g and most preferably from 1.5 g to 2 g.

For a composition intended to the animal consumption, specifically a non human mammal, including dog, cat or horse, a nutritional composition according to the invention is adapted to a daily administration of onion purified dry extract, provided by said composition, ranging from 1 to 10 g, preferably from 1 to 5 g of said extract.

In a still further aspect, the above nutritional composition may comprise other nutritional compounds, in combination with the onion purified dry extract.

Thus, a nutritional composition according to the invention may also comprise a calcium source, for example in the form of a physiologically acceptable organic or inorganic compound, such as inorganic calcium salts (calcium chloride, calcium phosphate, calcium sulfate, calcium oxide, calcium hydroxide or calcium carbonate) or calcium-containing organic components such as skim milk powder, calcium caseinate or calcium organic salts (calcium citrate, calcium maleate or mixtures thereof).

A nutritional composition according to the invention may also comprise vitamins, such as vitamin A, vitamin D, vitamin E, vitamin K, vitamin C, folic acid, thiamine, riboflavin, vitamin B₆, vitamin B₁₂, niacin, biotin or pantothenic acid.

A nutritional composition according to the invention may also comprise mineral nutrients and trace elements such as sodium, potassium, phosphorous, magnesium, copper, zinc, iron, selenium, chromium and molybdenum.

It may also comprise soluble fibers such agar, alginate, carob, carrageenan, acacia gum, guar gum, karaya gum, pectin or xanthan gum, these soluble fibers being in a hydrolyzed or non hydrolyzed form.

It may also comprise energetic compounds, in low amounts so as not to induce an hypercaloric diet, in particular one or more carbohydrate sources selected from maltodextrins, starch, lactose, glucose, sucrose, fructose, xylitol and sorbitol.

Moreover, a nutritional composition according to the invention may also comprise natural or artificial flavours, for example fruit flavours such as banana, orange, peach, pineapple or raspberry, or other plant flavours as vanilla, cocoa, coffee, etc.

A nutritional composition according to the invention may be in the powdered form. It also may present in the form of hard gelatin capsules containing such powder, tablets or liquid concentrates or syrups. It may also be incorporated into current consumption food such as in 4th range fresh products such as ready-to-eat salads; 5th range products; purées, prepared food; soups; vegetable juices (tomato, carrot . . . ); dairy products (fresh cheese . . . ); seasonings: vinegar, vinaigrette or aromatic mixtures.

As already previously mentioned, a composition intended to control the weight gain according to the invention may also present in the form of a pharmaceutical composition, as will be described hereafter.

### Pharmaceutical Compositions

The pharmaceutical composition may be a medical or a veterinary composition, in particular for dogs or cats, or horses.

A pharmaceutical composition intended to control the weight gain that is prepared from a purified dry onion extract, such as defined hereabove, is a form adapted to the oral, parenteral or intravenous administration. The oral forms are particularly preferred.

In its form intended for the administration to humans, a pharmaceutical composition made according to the invention advantageously comprises a purified dry onion extract amount adapted to a daily administration of said purified dry onion extract, delivered by said composition, ranging from 0.1 g to 10 g of purified dry onion extract.

In its form intended for the administration to animals, a pharmaceutical composition made according to the invention comprises a purified dry onion extract amount adapted to a daily administration of said extract, delivered by said composition, ranging from 0.5 g to 10 g.

A pharmaceutical composition such as defined hereabove comprises the onion purified dry extract in combination with at least one excipient selected in the group consisted of pharmaceutically acceptable excipients.

Methods for preparing pharmaceutical compositions may be easily found by the man skilled in the art, for example in Remington's Pharmaceutical Sciences, Mid. Publis. Co, Easton, Pa., USA.

Physiologically acceptable adjuvants, vehicles and excipients are also described in “Handbook of Pharmaceutical Excipients, second edition, American Pharmaceutical Association, 1994”.

To formulate a pharmaceutical composition according to the invention, the man skilled in the art will advantageously refer to the last edition of the European Pharmacopoeia or to the United States Pharmacopoeia (USP).

The man skilled in the art in particular will advantageously refer to the fourth edition (2002) of the European Pharmacopoeia, or to the 25-NF 20 edition of the United States Pharmacopoeia (USP).

The present invention also related a method for controlling the weight gain of a patient, said method comprising a step during which a therapeutically effective amount is given to the patient of an onion purified dry extract such as defined in the present description or of a pharmaceutical composition such as defined hereabove.

A pharmaceutical composition prepared according to the invention is in a liquid form or, more preferably, in a solid form.

For an oral administration, a solid pharmaceutical composition will be preferred, coming for example as tablets or capsules.

In the liquid form, a pharmaceutical composition coming as an aqueous suspension will be preferred.

Solid pharmaceutical forms may comprise as vehicles, adjuvants or excipients, at least one diluent, one flavouring agent, one solubilizing agent, one lubricant, one suspending agent, one disintegrating agent and one capsulating agent, the identity and function of these different traditional ingredients being fully documented in the European Pharmacopoeia or the United States Pharmacopoeia (USP).

Such compounds include for example magnesium carbonate, magnesium stearate, talc, lactose, pectin, dextrin, starch, gelatin, cellulose-containing materials, etc.

Compositions in a liquid form may also comprise water, where appropriate in admixture with propylene glycol or polyethylene glycol, as well as optional colouring agents, flavours, stabilizers and thickeners.

Moreover, the invention will be illustrated using the figure and the appended examples, without being limited thereto.

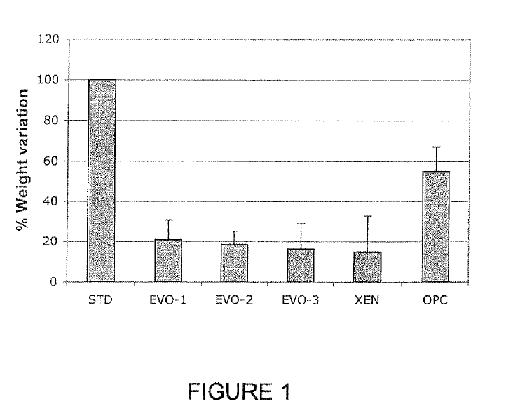

FIG. 1 illustrates the results concerning the weight evolution for hamsters consuming various diets, i.e.: STD (standard atherogenic diet), EVO-1 (0.06% of onion extract), EVO-2 (0.10% of onion extract), EVO-3 (0.15% of onion extract), XEN (0.03% of Xénical®), OBW (0.15% of Mincigrap®).

### EXAMPLES

### A. Material and Methods

### A.1. Selecting the Test Product Amounts

The only drug that represents a “digestive lipase inhibitor” available on the market and obtainable with a medical prescription is the Xénical® (Laboratoire Roche). Its active ingredient, “tetrahydrolipstatin” does specifically inhibit the pancreatic lipase, which results for the human species in an average reduction of about one third of the lipids contained in food (Zhi and al. 1994), and thus contributes to a weight loss (Sjöström and al. 1998). Many food complements, phytotherapy-derived products are also proposed for initiating and/or supporting the weight loss.

In the present study, the effects of the onion extract EV06/05/02 were compared to those of the product marketed on the trade name “Mincigrap®” by the Laboratoires Arkopharma. Indeed, “Mincigrap®” represents a concentrated source of polyphenols derived from grape-cakes which impact on the digestive enzymes have been demonstrated in vitro (Griffiths 1986, Tebib and al. 1994, Carmona 1996) and in vivo in rats (Horigome and al. 1988, Vallet and al. 1994).

The persons who have an excess weight (BMI≧25 kg/m²) or that are obese (BMI≧30 kg/m²) and who wish to loss weight are recommended to reduce their caloric intake in a less or more pronounced way (from 1400 to 2200 kCal/day) in the context of a balanced diet (providing from 45 to 75 g/day of lipids). The Xénical®, the “Mincigrapo®” and of the onion extract EV06/05/02 doses to administrate to the hamsters were determined from these data, that are given hereunder in Table 1.

TABLE 1
Dosage regimen and amounts of Xénical ® or
“Mincigrap ®” as compared to the body weight
and to the food lipids ingested by humans.

| | | Dose in mg/kg | |
|---|---|---|---|
| Name of the | | BW | |
| proprietary | | (from 60 to 120 | Dose in mg/g TG |
| drug | Dosage regimen | kg) | (45-75 g of lipids) |
| Xénical ® | 3 × 240 mg capsule | 6 to 12 | 10 to 16 |
| | 720 mg/day | | |
| “Mincigrap ®” | 6 × 390 mg capsule | 20 to 40 | 31 to 52 |
| | 2340 mg/day | | |

Since the study was made on an animal model of the non-obese type rodent, but that nevertheless did receive a lipid-rich diet, the following product doses were tested:
for Xénical®: 12.5 mg/kg of body weight,
for “Mincigrap®”: 50 mg/kg of body weight.
   The EV06/05/02 onion extract amounts according to the invention are chosen on the one hand so that they are slightly higher than those of Xénical®, which is a drug, hence not a food complement, and on the other hand, so that they make the comparison possible with the “Mincigrap®”, which is a polyphenol source:
1st test dosage: 30 mg/kg of body weight,
2d test dosage: 50 mg/kg of body weight,
3rd test dosage: 75 mg/kg of body weight.

### A.2 Animals and Diets

The golden hamster, strain Aura Rj was chosen as an animal model (Janvier breeder).

Male hamsters weighting between 83 and 113 grams at the beginning of the study were placed in cages of 4 individuals at most, in a air-conditioned room at 22° C. After 10 days of adaptation to the animal space, the hamsters were distributed into 6 groups and were fed for 14 days either with a standard lipid-poor food (control group), or with a “test diet”, that is to say an atherogenic diet containing either the onion extract, or Xénical®, or “Mincigrap®”:
Control group or “STD” (n=8): aliment d'entretien for hamsters
EV0-1 Group (n=8): atherogenic diet containing 0.06% of EV06/05/02 extract.
EV0-2 Group (n=8): atherogenic diet containing 0.10% of EV06/05/02 extract.
EV0-3 Group (n=8): atherogenic diet containing 0.15% of EV06/05/02 extract.
XEN group (n=8): atherogenic diet containing 0.03% of Xénical®.
MCG group (n=8): atherogenic diet containing 0.15% of “Mincigrap®”.

The control diet is a maintenance feeding diet for hamsters UAR04 (rational feeding plant, Villemoisson-sur-Orge, France), wich calorific value=3400 kcal/kg.

The atherogenicity of the “test diets” is obtained by complementing in cholesterol (0.25%) and in lipids (10%) which are provided by olive oil (½), sunflower oil (¼) and tallow (¼). Said “test diets” are isoproteic, isoglucidic and isolipidic in nature and have a calorific value of 3620 kcal/kg. These “test diets” were prepared by Mucedola SRL (Settimo Milanese, Italy) according to the formulation given in Table 2 hereunder.

TABLE 2
“test diets” compositions

| Ingrédients g/for 100 g | | | | | |
|---|---|---|---|---|---|
| diet | EV0-1 | EV0-2 | EV0-3 | XEN | MCG |
| EV06/05/02 | 0.06 | 0.10 | 0.15 | — | — |
| Xénical ® | — | — | — | 0.03 | — |
| “Mincigrap ®” | — | — | — | — | 0.10 |
| Cholesterol | 0.25 | 0.25 | 0.25 | 0.25 | 0.25 |
| Lipids | 10.0 | 10.0 | 10.0 | 10.0 | 10.0 |
| Saccharose | 20.00 | 20.00 | 20.00 | 20.00 | 20.00 |
| Starch | 24.00 | 24.00 | 24.00 | 24.00 | 24.00 |
| Cellulose | 5.00 | 5.00 | 5.00 | 5.00 | 5.00 |
| Proteins | 24.00 | 24.00 | 24.00 | 24.00 | 24.00 |
| Vitamins | 1.25 | 1.25 | 1.25 | 1.25 | 1.25 |
| Minerals | 5.00 | 5.00 | 5.00 | 5.00 | 5.00 |
| Water | 10.44 | 10.40 | 10.35 | 10.47 | 10.40 |

### A.3 Data Processing

The statistical data processing is performed with the Stat-View V software, marketed by SAS Institute Inc., Cary N.C., USA.

The results (weight and weight variations, ingested portions, absorption percentages, lipemia values and lipoprotein oxydability tests) are expressed as a mean value ±SEM (standard error of the mean) and are all presented in the appendix (paragraph 6).

For a given parameter, the statistically significant differences between the mean values of the various groups are determined by the variance analysis (ANOVA) for non repeated measures at the probability of P<0.05.

The absorption coefficient is calculated as follows:

$$\% absorption = ( 1 - \frac{total { }^{14} C in feces \text{/} total {}^{3} H in feces }{total {}^{14} C in the bolus \text{/} total {}^{3} H in the bolus} ) \times 100$$

### A.4. Qualitative and Quantitative Analysis of the Onion Dry Extract.

A qualitative and quantitative analysis of an onion dry extract according to the invention is detailed in the Table below.

| | Polyphenols |
|---|---|
| Molecules | (g/100 g of dry extract) |
| Quercetin-3,4′-diglucoside | 1.63 |
| Isorhamnetin-3,4′-diglucoside | 0.41 |
| Other quercetin diglucosides | 1.60 |
| Quercetin 3-monoglucoside | 0.57 |
| Quercetin-4′-monoglucoside | 18.89 |
| Isorhamnetin-4′-monoglucoside | 0.21 |
| Quercetin | 9.99 |
| Isorhamnetin | 0.19 |
| Kaempferol | 0.13 |
| Total flavonols | 33.62 |

### B. Example 1

### Use of a Composition Comprising a Flavonol-Rich Onion Purified Extract for Controlling the Weight Gain

### B.1. Weight Evolution and Ingested Portions

### B.1.1. Weight Evolution

Hamsters in each group were weighted at the beginning (D1) and at the end of the experiment (D14). The weight average values at D1 and at D14 are given in Table 3, hereunder. Weight variations between D1 and D14 are given in FIG. 1.

TABLE 3
Weight average values (grams) for the hamsters in each group at D1 and D14.

| Groups | STD | EV0-1 | EV0-2 | EV0-3 | XEN | MCG |
|---|---|---|---|---|---|---|
| Weight at D1 (g) | 92.9 ± 2.1 | 102.5 ± 1.7 | 101.8 ± 1.6 | 104.6 ± 1.5 | 105.3 ± 1.6 | 103.4 ± 2.3 |
| Weight at D14 (g) | 101.3 ± 1.9 | 104.3 ± 1.9 | 103.4 ± 1.6 | 106.0 ± 1.4 | 106.5 ± 2.5 | 108.0 ± 2.3 |

STD versus EV0-1, EV0-2, EV0-3, XEN (P < 0.0001), MCG (P < 0.01) MCG versus EV0-1, EV0-2, EV0-3, XEN (P < 0.05)

Compared to the 5 test groups, the STD group increased significantly in weight (8.4±0.5 g versus 1.25±1.49 g for the XEN group and 4.62±1.03 g for the MCG group). The hamsters in the 3 EV0 groups and in the XEN group did significantly gain less weight than those belonging to the MCG group (2.6 to 3.3 times as much).

Finally, no dose-dependent effect on weight gain could be evidenced for the EV06/05/02 extract.

Bibliographic data do confirm that the weight evolution for the STD group does coincide with that of this animal model (it is hence possible to do remarks about the effects of the test products on this parameter).

Many nutritional interventions conducted in the golden hamster fed with a lipid-rich food (from 12 to 20% depending on the studies) enabled to evaluate the effects of such diets on the weight gain. This results are given in Table 4, hereunder:

TABLE 4
Hamster weight average values (grams)
for each group at D1 and D14.

| References | Amount and nature of the lipids | Weight gain(g/d) |
|---|---|---|
| Kurowka & | 12% of lipids provided by | 0.60 ± 0.22 and 0.62 ± |
| Manthey | coconut oil (10%) and safflower | 0.24 |
| (2004) | oil (2%) and 0.1% of cholesterol | |
| Auger and | 15% of lipids, provided by lard, | Estimated to range |
| al. (2004) | and 0.5% of cholesterol | from 0.38 to 0.63 |
| | | depending on the initial |
| | | weight of the hamsters |
| Nicolisi and | 20% of lipids, monounsaturated | 0.71 (oleic acid) and |
| al. (1998) | fatty acid source (oleic acid) | 0.68 (myristic acid) |
| | and saturated fatty acid | |
| | source (myristic acid) | |
| Results of | 10% of lipids (sugar mixture) | 0.13 (EV0-1), 0.11 |
| the present | and 0.25% of cholesterol | (EV0-2), 0.10 (EV0-3), |
| study | | 0.09 (XEN) and 0.33 |
| | | (MCG). |

The analysis of these results is interesting since it does reveal that hamsters fed with EV06/05/02 onion extract-, Xénical®- or “Mincigrap®”-containing diets have a very poor average weight gain (g/d) considering the lipid enrichment of these diets (10% versus 4% for the maintenance feeding diet).

However, in the studies of Auger and al. (2004), Kurowka & Manthey (2004), as well as in the study of Wiseman and al. (2002), the enrichment of such diets with polyphenols (powdered polyphenols derived from grape seeds and grape-cakes, polyphenols derived from citrus fruits, and water-soluble olive polyphenols, respectively) showed no effect on the weight gain. These results do suggest that the effects on the weight gain observed in this study could be specific to the onion extract, and probably to the flavonols (quercetin, isorhamnetin and Kaempferol) contained therein. For the human as for the animal, there are little data establishing a correlation between a polyphenol intake, except tea polyphenols, that is to say essentially catechins, and a weight maintain or a weight loss. However, there isn't any information about an effect of flavonols, in particular of quercetin or quercetin glycosides on the weight gain.

Finally, concerning the Xénical® activity, a study was conducted on rats that were made obese and then received Xénical® (54 mg/kg of body weight) which showed that the weight loss of these rats is of 8% (Hogan and al. 1987). In another study conducted on non obese rats, the use of 2 mg of Xénical® per gram of lipids (3 mg/g of lipids in the present study), did reduce the weight gain by 81% (Ackroff & Sclafani, 1996). This result seems to more correlate with the Xénical® effect on the weight evolution demonstrated by the present study: weight variation in hamsters treated with Xénical® compared to non treated hamsters (the STD qroup) corresponds to 85%.

### B.1.2. Ingested Portions and Test Product Dosage

The average ingested food amounts for each hamster in a day were measured by weighting the distributed granules and the residual granules at the beginning and at the end of each nutritional intervention. These results enable to calculate the mean ingested energetic amount in a day as well as the onion extract doses, the Xénical® (XEN) or the “Mincigrap®” (MCG) doses that were actually administered (see Table 5 below).

TABLE 5
Total ingested energetic amounts (kcal per kg of body weight in a day) and
doses of test products actually administered (mg per kg of body weight).

| | STD | EV0-1 | EV0-2 | EV0-3 | XEN | MCG |
|---|---|---|---|---|---|---|
| Total | 191.8 ± 4.0 | 164.7 ± 2.7 | 168.2 ± 2.6 | 165.9 ± 2.3 | 186.5 ± 2.7 | 184.8 ± 4.1 |
| energetic | | | | | | |
| amount | | | | | | |
| (kCal/kg/d) | | | | | | |
| Test product | — | EV06/05/02 | EV06/05/02 | EV06/05/02 | Xénical ® | “Mincigrap ®>> |
| doses | | 27.0 ± 0.5 | 46.0 ± 0.8 | 69.0 ± 0.9 | 15.4 ± 0.3 | 49.8 ± 1.0 |
| (mg/kg BW) | | | | | | |

Hamsters belonging to the XEN and MCG groups have an energetic consumption comparable to that of the STD group whereas hamsters belonging tot the EV0 groups have a lower consumption (−14% to −12% for the EV0-1 and EV0-2 groups, respectively). In the same way, the ingested portion for the XEN group is similar to that of the MCG group (<1%) but remains significantly higher than that of the EV0-1 (11.7%), EV0-2 (11.0%) and EV0-3 (9.8%) groups.

Compared to the bibliographic sources the amounts (on average 4.96±0.15 g/day) that were ingested by the hamsters of this study do correlate with those of the studies carried out under similar experimental conditions, that is to say using either a maintenance feeding diet for hamster (Gilat and al. 2003), or a lipid-enriched food (see references in Table 5). In this latter case, the ingested portions do vary from 4.60±0.10 g/d (Auger and a/2004) to 7.6±0.8 g/d (Kurwska & Manthey 2004) and to 9.8±0.3 g/d (Nicolosi and al. 1998).

### B.1 Section Conclusion:

The EV06/05/02 onion extract does significantly limit the weight gain as compared to a standard diet and as compared to the powdered grape-cake “Mincigrap®”, used with the same dosage. The efficiency was maximal already at the smallest dose (27 mg/kg BW) and could be compared with that of Xénical® (15 mg/kg BW).

As opposed to the research results for the Xénical® effects on animals, no study did mention any weight gain limiting effect by chronical ingestion of polyphenols.

### B.2. Conclusion

### B.2.1. EV06/05/02 Extract Effects as Compared to the Standard Diet

The EV06/05/02 extract does remarkably act on the weight gain as compared to the standard diet. Thus, hamsters did gain from 6.7, 4.8 and 5.4× less weight when fed with a food supplemented with the EV06/05/02 extract at doses of 27, 46 and 69 mg/kg of body weight, respectively.

However, it should be noted that while hamsters fed with diets based on the EV06/05/02 extract did present a caloric intake as related to their body weight lower than that of the standard group, such difference remains poor (i.e. 12 to 14%) and cannot be responsible for the weight loss.

The results of the study do confirm that the EV06/05/02 onion extract reduces the food triglyceride assimilation in vivo and show the positive effects of this extract on the weight gain. While no dose-dependent effect could be clearly demonstrated, it is important to emphasize the efficiency of this extract with a minimum dosage (27 mg/kg of body weight or 6 mg/g of triglycerides).

B.2.2. EV06/05/02 Extract Versus Xénical®

The effect of the EV06/05/02 extract on the weight gain is comparable to that of Xénical®. These results clearly show that the EV06/05/02 extract used at dosages of 27 mg/kg BW (that is to say of twice as much as Xénical®) is as efficient as Xénical® for limiting the weight gain.

These results do suggest that the action of the EV06/05/02 extract on the weight gain would not be, or in any case would not only be associated with the lipid assimilation inhibition and that other mechanisms characteristic of the onion extract components would be implied. This hypothesis is interesting when considering that excessively inhibiting the lipid digestion, such as induced by Xénical®, causes side effects for the patient comfort (nausea and vomiting, diarrhea, abdominal pain, etc.), and in the longer term, has negative effects on the vitaminic status.

The EV06/05/02 onion extract, whatever the test dosage, has an efficiency on the weight gain comparable to that of Xénical® although the triglyceride absorption is less affected.

### B. 2.3. EV06/05/02 Extract Versus “Mincigrap®”

Despite the lower effect on the food lipid absorption, the EV06/05/02 onion extract does more efficiently and more significantly limit the weight gain as compared to the “Mincigrap®”: by a factor of 3 at equivalent dose (46 mg/kg BW for EV06/05/02; 49 mg/kg BW for “Mincigrap®”), by a factor of 2.6 for the 27 mg/kg BW dose and by a factor of 3.4 for the 69 mg/kg BW dose.

As compared to <<Mincigrap®”, the efficiency on the weight gain of the EV6/05/02 extract, whatever the dosage, is substantially higher, while the fecal excretion of the food lipids is lower.

The results of this study show that the onion extract does act on the weight gain. Although the results are not presented therein, it was demonstrated that the onion extract according to the invention does act on the weight gain without inducing any drastic triglyceride absorption inhibition in the hamster. This observation is interesting as a moderate effect on the lipid digestion (and probably of the other macronutrients) should attenuate micronutrient bioavailability disorders (fat-soluble and water-soluble vitamins, minerals and trace elements), and hence should limit deficiency risks.

Since the efficiency on the weight evolution is comparable to that of Xénical® and higher than that of “Mincigrap®”, both inducing an increased fecal excretion of the food lipids, it can be considered that the onion extract acts on this parameter via other mechanisms that might be further investigated.

However, comparing the onion extract effects on the weight gain with those of Xénical® and “Mincigrap®” makes it possible to compare the extract activity against a lipase specific inhibitor, the effects of which have been confirmed on humans and against a digestive enzyme non specific inhibitor.

As a conclusion, the results of example 1 show that EV06/05/02 onion extract is active on the weight loss at an acceptable dosage as regards the nutritional and physiological point of view in the hamster (from 27 to 69 mg/kg of BW), what would correspond for the human species to a daily ingested amount of from 1.5 to 2 grams. Such a property would justify the use of onion extract as an “aid for weighting loss” in humans.

### REFERENCES

Zhi J, Melia A T, Guerciolini R, Chung J, Kinberg J, Hauptman J B, Patel I H. Retrospective population-based analysis of the dose-response (fecal fat excretion) relationship of orlistat in normal and obese volunteers. Clin Pharmacol Ther 1994 July; 56(1):82-5
Sjöström L, Rissanen A, Andersen T, Boldrin M, Golay A, Koppeschaar H P, Krempf M. (1998). Randomised placebo-controlled trial of orlistat for weight loss and prevention of weight regain in obese patients. European Multicentre Orlistat Study Group. Lancet July 18; 352(9123): 167-72.
Griffiths D W. The inhibition of digestive enzymes by polyphenolic compounds. Adv Exp Med Biol. 1986; 199:509-16.
Carmona A. Tannins: thermostable pigments which complex dietary proteins and inhibit digestive enzymes. Arch Latinoam Nutr. 1996 December; 44(4 Suppl 1):31 S-35S.
Tebib K, Rouanet J M, Besancon P. Effect of grape seed tannins on the activity of some rat intestinal enzyme activities. Enzyme Protein. 1994-95; 48(1):51-60.
Auger C, Gerain P, Laurent-Bichon F, Portet K, Bornet A, Caporiccio B, Cros G, Teissedre P L, Rouanet J M. Phenolics from commercialized grape extracts prevent early atherosclerotic lesions in hamsters by mechanisms other than antioxidant effect. J Agric Food Chem. 2004 Aug. 11; 52(16):5297-302.
Kurowska E M, Manthey J A. HypoLipidmic effects and absorption of citrus polymethoxylated flavones in hamsters with diet-induced hypercholesterolemia. J Agric Food Chem. 2004 May 19; 52(10):2879-86.
Wiseman S A, Tijburg L B, van of Put F H. Olive oil phenolics protect LDL and spare vitamin E in the hamster. Lipids. 2002 November; 37(11):1053-7.
Gilat T. Leikin-Frenkel A, Goldiner I, Juhel C, Lafont H, Gobbi D, Konikoff F M. Prevention of diet-induced fatty liver in experimental animals by the oral administration of a fatty acid bile acid conjugate (FABAC). Hepatology. 2003 August; 138(2):436-42.
Nicolosi R J, Wilson T A, Rogers E J, Kritchevsky D. Effects of specific fatty acids (8:0, 14:0, cis-18:1, trans-18:1) on plasma lipoproteins, early atherogenic potential, and LDL oxidative properties in the hamster. J Lipid Res. 1998 October; 39(10): 1972-80.

1. An onion extract weight gain control composition comprising from 20% to 40% by weight of polyphenols belonging to the flavonol family, said onion extract having following composition characteristics, expressed in weight based on the dry extract total weight:
from 60% to 90% by weight of the total flavonols in said extract consist in glycosylated polyphenols,
from 85% to 98% by weight of the total flavonols in said extract consist in quercetin in a free form (aglycone) or in a glycosylated form,
from 5% to 20% by weight of the total flavonols in said extract consist in quercetin-3,4′-diglucoside,
from 30% to 70% by weight of the total flavonols in said extract consist in quercetin-3-monoglucoside and quercetin-4′-monoglucoside, and
from 20 to 30% by weight of the total flavonols in said extract consist in quercetin.

2. The composition according to claim 1, characterized in that, in said onion extract, from 70% to 80% by weight of total polyphenols in said extract consist in glycosylated flavonols.

3. The composition according to claim 1, characterized in that, in said onion extract, from 90% to 95% by weight of the total flavonols in said extract represent a quercetin in a free form (aglycone) or in a glycosylated form.

4. The composition according to claim 1, characterized in that, in said onion extract, from 7% to 13% by weight of the total flavonols in said extract consist in quercetin-3,4′-diglucoside.

5. The composition according to claim 1, characterized in that, in said onion extract, from 40% to 60% by weight of the total flavonols in said extract consist in quercetin-3-monoglucoside and quercetin-4′-monoglucoside.

6. The composition according to claim 1, characterized in that said onion extract comprises from 25% to 35% by weight of polyphenols belonging to the flavonol family.

7-8. (canceled)

9. A nutritional composition for controlling weight gain comprising:
the onion extract weight gain control composition of claim 1, and a nutritional compound.

10. A pharmaceutical composition for controlling weight gain comprising:
the onion extract weight gain control composition of claim 1, and a pharmaceutically acceptable excipient.

11. A food composition for controlling weight gain comprising:
the onion extract weight gain control composition of claim 1, and an energetic compound.

12. A method of controlling weight gain in an individual comprising:
administering to an individual an effective amount of the onion extract weight gain control composition of claim 1, to control weight gain in the individual.
